(19) United States
(12) Patent Application Publication
(10) Pub. No.: US 2023/0010449 A1
(43) Pub. Date: Jan. 12, 2023

(54) Inhalation Device

(71) Applicant: JT International SA, Geneva (CH)

(72) Inventors: Andrew Robert John Rogan, Forres Central Scotland (GB); Gordon Montgomery, Ballymena Co. Antrim (GB); Kyle Adair, Lisburn, Antrim (GB); Akihiko Suzuki, Tokyo (JP)

(73) Assignee: JT International SA, Geneva (CH)

(21) Appl. No.: 17/779,658

(22) PCT Filed: Nov. 24, 2020
(86) PCT No.: PCT/IB2020/061064

§ 371 Date: May 25, 2022

(30) Foreign Application Priority Data
Nov. 25, 2019 (EP) 19211318.1

(51) Int. Cl. A24F 40/51; A24F 40/53; A24F 40/57

(52) U.S. Cl. CPC A24F 40/51; A24F 40/53; A24F 40/57; A61M 2205/3313; A61M 2205/3317; A61M 2205/3324; A61M 2205/6018

(57) ABSTRACT

An inhalation device defines a receptacle for receiving an aerosol-forming substrate containing at least one vaporizable substance; an atomizer configured to generate an aerosol from the aerosol-forming substrate; a first sensor configured to generate a first signal or first data associated with a presence of at least one chemical substance in the generated aerosol; a second sensor configured to generate a second signal or second data associated with a further characteristic of the generated aerosol or of the aerosol-forming substrate or of the at least one vaporizable substance; and at least one controller configured to separately analyze both the first signal or first data and the second signal or second data to determine first and second analysis results, and configured to determine an operation mode of the inhalation device or to determine whether to alter an operation of the inhalation device based on both the first and second analysis results.

### FIELD OF THE INVENTION

The present disclosure relates to inhalation devices for producing an aerosol or vapor for inhalation by a user. The present disclosure also relates to inhalation device control methods.

### BACKGROUND

GB2524779 and US2014/0345633 disclose an inhalation device containing a plurality of sensors. GB2524779 discloses using sensor measurements to deliver a predetermined dosage of different active chemicals to a user. US2014/0345633 discloses the use of a temperature sensor to maintain a heater element below a predetermined temperature so as to avoid certain volatile compounds being released by a heated aerosol-forming substrate.

US2017/0224024 discloses an aerosol-generating device containing a heater, a combustion gas detector and a controller. The controller determines a combustion gas level and compares it to a threshold level. When the measured combustion gas level is above a first threshold level, the controller reduces the supply of power to the heater. When the measured combustion gas level is above a second threshold level, the controller activates an indicator on the device. When the measured combustion gas reaches a stop level, the controller stops the supply of power to the heater.

Such known inhalation devices measure a characteristic related to the functioning of the inhalation device and implement a particular device operation or action on the basis of this measurement. An operation decision is taken on the basis of an isolated factor, for example, a combustion gas level of the generated vapor, a temperature of a heater element or an active chemical concentration. Given that decisions are taken on the basis of an isolated factor, the actions taken by inhalation devices on the basis of an isolated factor provide limited assurance that the action actually taken is sufficient to assure objectives, such as, assuring user and device safety. Potential risks to user and device safety can come from multiple elements of the inhalation device or inter-related elements of the inhalation device.

Consequently, the background art presents a number of deficiencies and problems and the present disclosure seeks to address these difficulties.

### SUMMARY

It is therefore one aspect of the present disclosure to provide an inhalation device. Preferably, the inhalation device comprises a receptacle for receiving an aerosol-forming substrate containing a vaporizable substance, an atomizer configured to generate an aerosol from the aerosol-forming substrate, a first sensor configured to generate a first signal or first data associated with a presence of at least one chemical substance in the generated aerosol, at least a second sensor configured to generate a second signal or second data associated with a further characteristic of the generated aerosol or of the aerosol-forming substrate or of the vaporizable substance, and at least one controller configured to separately analyze both the first signal or first data and the second signal or second data to determine first and second analysis results, and configured to determine an operation action or mode of the inhalation device or to determine whether to alter an operation of the inhalation device based on both the first and second analysis results.

The inhalation device of the present disclosure takes into account multiple factors and independent information from multiple independent sensors to take a more informed decision to thus provide increased assurances as to user safety or device protection.

According to an aspect of the present disclosure, the at least one controller is configured to determine an operation action or mode of the inhalation device or to determine whether to alter an operation of the inhalation device based on a conjunction of the first and second analysis results.

According to another aspect of the present disclosure, the at least one controller is configured to determine an operation mode or action of the inhalation device or to determine whether to alter an operation of the inhalation device based on a separate evaluation of both the first and second analysis results.

According to another aspect of the present disclosure, the at least one controller is configured to independently evaluate the first and second analysis results.

According to another aspect of the present disclosure, the at least one controller is configured to analyze the first signal or first data independently of the second signal or second data.

According to another aspect of the present disclosure, the at least one controller is configured to determine an operation mode or action of the inhalation device or to determine whether to alter an operation of the inhalation device based on a combination of the first and second analysis results.

According to another aspect of the present disclosure, the at least one controller is configured to analyze the second signal or second data to determine an identity of the aerosol-forming substrate or of the vaporizable substance, or to validate the aerosol-forming substrate, substance or generated aerosol as suitable for the inhalation device.

According to another aspect of the present disclosure, the at least one controller is configured to analyze the second signal or second data to determine whether an operation condition or operation behavior of the inhalation device is in conformity with a predetermined operation condition or operation behavior, or to determine whether an inhalation device element is located in a predefined position in or on the inhalation device.

According to another aspect of the present disclosure, the at least one controller is configured to determine an operation mode or action of the inhalation device or to determine whether to alter the operation of the inhalation device based on both (i) the at least one chemical substance in the generated aerosol detected by the first sensor and (ii) a determined identity of the aerosol-forming substrate or of the vaporizable substance determined using the second sensor, or validation or non-validation of the aerosol-forming substrate, substance or generated aerosol as suitable for the inhalation device, or an operation condition or operation behavior of the inhalation device being in conformity with a predetermined operation condition or operation behavior, or an inhalation device element being located in a predefined position in or on the inhalation device.

According to another aspect of the present disclosure, the at least one controller is configured to permit operation of the atomizer or permit continued operation of the atomizerwhen (i) the at least one chemical substance in the generated aerosol detected by the first sensor is determined to be a conditionally or provisionally authorized chemical substance and (ii) a determined identity of the aerosol-forming substrate or of the vaporizable substance corresponds to an authorized aerosol-forming substrate or authorized vaporizable substance, or the aerosol-forming substrate, vaporizable substance or generated aerosol is validated as being suitable for the inhalation device, or an operation condition or operation behavior of the inhalation device is in conformity with a predetermined operation condition or operation behavior, or an inhalation device element is located in a predefined position in or on the inhalation device.

According to another aspect of the present disclosure, the at least one controller is configured to determine (a) whether the at least one chemical substance in the generated aerosol detected by the first sensor is a conditionally or provisionally authorized chemical substance and (b) whether a determined identity of the aerosol-forming substrate or the vaporizable substance corresponds to an authorized aerosol-forming substrate or authorized vaporizable substance, or whether the aerosol-forming substrate, aerosol-forming substance or generated aerosol is validated as being suitable for the inhalation device, or whether an operation condition or operation behavior of the inhalation device is in conformity with a predetermined operation condition or operation behavior, or an inhalation device element is located in a predefined position in or on the inhalation device.

According to another aspect of the present disclosure, the second sensor is configured to directly contact the aerosol-forming substrate to perform a measurement to generate the second signal or second data.

According to another aspect of the present disclosure, the first analysis result concerns user safety, and the second analysis result concerns aerosol-forming substrate identification, or vaporizable substance identification, or validation of the aerosol-forming substrate, substance or generated vapor as suitable for the inhalation device.

According to another aspect of the present disclosure, the inhalation device further includes an aerosol chamber for receiving the generated aerosol, an outlet and a flow channel located between the aerosol chamber and the outlet.

According to another aspect of the present disclosure, the first and second sensors are configured to communicate with the at least one controller to respectively provide the first signal or first data and the second signal or second data thereto.

According to another aspect of the present disclosure, the first sensor is at least partially located in a flow path of the generated aerosol extending between the aerosol chamber and the outlet.

According to another aspect of the present disclosure, the first sensor comprises or consists solely of a combustion gas sensor configured to detect the presence of at least one chemical substance in the generated aerosol.

According to another aspect of the present disclosure, the second sensor comprises or consists solely of a temperature sensor, a chemical sensor, a mass sensor, a pH sensor, a photo-sensor, a Hall sensor, a capacitance sensor, an optical emission sensor, or a humidity sensor.

According to another aspect of the present disclosure, the temperature sensor is configured to measure temperature values or values representing temperature values of the aerosol-forming substrate or the generated aerosol, and to provide said values to the at least one controller.

According to another aspect of the present disclosure, the at least one controller is configured to analyze the temperature values to determine whether the temperature values are in conformity with a predetermined temperature profile.

According to another aspect of the present disclosure, the at least one controller is configured to permit operation of the atomizer or permit continued operation of the of the atomizer when (i) the at least one chemical substance in the generated aerosol is determined to be an authorized chemical substance and (ii) the temperature values are in conformity with a predetermined temperature profile.

According to another aspect of the present disclosure, the mass sensor is configured to measure mass values or values representing mass values of the aerosol-forming substrate and the vaporizable substance, and to provide said values to the at least one controller.

According to another aspect of the present disclosure, the at least one controller is configured to analyze the mass values to determine whether a predetermined change in mass has occurred during operation or heating of the aerosol-forming substrate.

According to another aspect of the present disclosure, the at least one controller is configured to permit operation of the atomizer or permit continued operation of the of the atomizer when (i) the at least one chemical substance in the generated aerosol is determined to be an authorized chemical substance and (ii) the mass values are in conformity with a predetermined change-in-mass profile.

According to another aspect of the present disclosure, the at least one controller is configured to analyze a measured pH value, a humidity value or a chemical sensor value to determine an identity of the aerosol-forming substrate.

According to another aspect of the present disclosure, the at least one controller is configured to permit operation of the atomizer when (i) the at least one chemical substance in the generated aerosol is determined to be an authorized chemical substance and (ii) the determined identity of the aerosol-forming substrate is an authorized identity.

According to another aspect of the present disclosure, the capacitance sensor is configured to measure a capacitance value or a value representing a capacitance value of at least a portion of the aerosol-forming substrate and the vaporizable substance, and to provide said value to the at least one controller.

According to another aspect of the present disclosure, the at least one controller is configured to analyze the capacitance value or the value representing a capacitance value to determine an identity of the aerosol-forming substrate and the vaporizable substance.

According to another aspect of the present disclosure, the at least one controller is configured to permit operation of the atomizer or permit continued operation of the of the atomizer when (i) the at least one chemical substance in the generated aerosol is determined to be an authorized chemical substance and (ii) the determined identity of the aerosol-forming substrate and the vaporizable substance is an authorized identity.

According to another aspect of the present disclosure, the optical emission sensor is configured to measure an optical emission signal emitted by at least a portion of the aerosol-forming substrate or the vaporizable substance or the generated aerosol under optical excitation, and configured to provide said signal to the at least one controller.

According to another aspect of the present disclosure, the at least one controller is configured to analyze the optical emission signal to determine an identity of the aerosol-forming substrate, the vaporizable substance or the generated aerosol.

According to yet another aspect of the present disclosure, the at least one controller is configured to permit operation of the atomizer or permit continued operation of the of the atomizer when (i) the at least one chemical substance in the generated aerosol is determined to be an authorized chemical substance and (ii) the determined identity of the aerosol-forming substrate or the vaporizable substance or the generated aerosol is an authorized identity.

According to another aspect of the present disclosure, the optical emission sensor comprises or consists solely of a fluorescence sensor, and the aerosol-forming substrate or the vaporizable substance contains fluorescing markers or chemicals to provide the optical emission.

According to another aspect of the present disclosure, the photo-sensor is configured to measure an optical intensity signal transmitted through at least a portion of the aerosol-forming substrate and vaporizable substance, and configured to provide said signal to the at least one controller.

According to another aspect of the present disclosure, the at least one controller is configured to analyze the optical intensity signal to determine an identity of the aerosol-forming substrate and vaporizable substance.

According to another aspect of the present disclosure, the at least one controller is configured to permit operation of the atomizer or permit continued operation of the of the atomizer when (i) the at least one chemical substance in the generated aerosol is determined to be an authorized chemical substance and (ii) the determined identity of the aerosol-forming substrate and the vaporizable substance is an authorized identity.

According to an aspect of the present disclosure, the inhalation device is an electronic cigarette.

According to another aspect of the present invention, inhalation device control method is provided, wherein

the inhalation device comprises a reservoir for receiving an aerosol-forming substrate containing a vaporizable substance, an atomizer configured to generate an aerosol from the aerosol-forming substrate, a first sensor configured to generate a first signal or first data associated with a presence of at least one chemical substance in the generated aerosol, at least a second sensor configured to generate a second signal or second data associated with a further characteristic of the generated aerosol or of the aerosol-forming substrate or of the vaporizable substance; and at least one controller. Preferably, the method includes the steps of separately analyzing, by the at least one controller, both the first signal or first data and the second signal or second data to determine first and second analysis results, and determining an operation mode or action of the inhalation device or determining whether to alter an operation of the inhalation device based on both the first and second analysis results.

The above and other objects, features and advantages of the present invention and the manner of realizing them will become more apparent, and the invention itself will best be understood from a study of the following description with reference to the attached drawings showing some preferred embodiments of the invention.

### BRIEF DESCRIPTION OF THE DRAWINGS

The accompanying drawings, which are incorporated herein and constitute part of this specification, illustrate the presently preferred embodiments of the invention, and together with the general description given above and the detailed description given below, serve to explain features of the invention.

FIG. 1 shows a schematic of an exemplary implementation of an inhalation device according to present disclosure.

FIG. 2 depicts a schematic of another exemplary implementation of an inhalation device according to present disclosure.

FIG. 3 shows a schematic of yet another exemplary implementation of an inhalation device according to present disclosure.

FIG. 4 shows a schematic of another exemplary implementation of an inhalation device according to present disclosure.

FIG. 5 shows a schematic of further exemplary implementation of an inhalation device according to present disclosure.

FIG. 6 schematically shows exemplary processing by a controller of an inhalation device of the present disclosure.

Herein, identical reference numerals are used, where possible, to designate identical elements that are common to the Figures. Also, the images are simplified for illustration purposes and may not be depicted to scale.

### DETAILED DESCRIPTION OF SEVERAL EMBODIMENTS

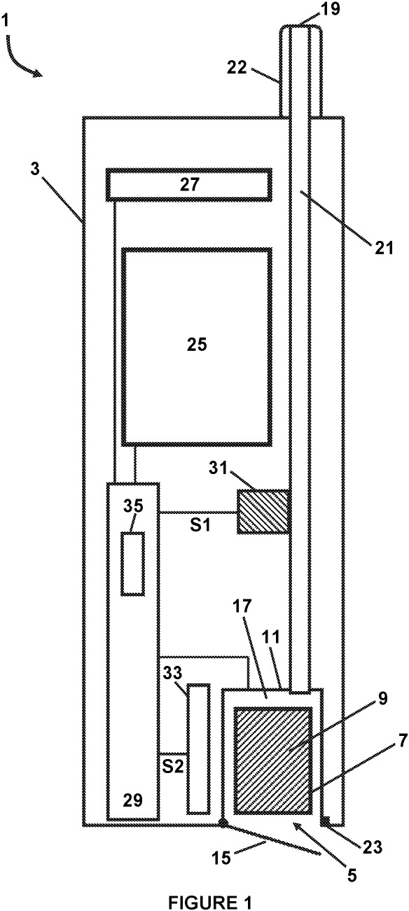

Exemplary embodiments of an inhalation device according to the present disclosure are shown in FIGS. 1 to 5. The Figures depict exemplary schematic views of the inhalation device or vapor generating device with different elements in a symbolic representation.

The inhalation device 1 can, for example, comprise or consist of an electronic cigarette.

The inhalation device 1 includes a housing 3, a receptacle 5 for receiving an aerosol-forming substrate 7 containing at least one vaporizable substance 9, and an atomizer 11 configured to generate an aerosol or vapor from the aerosol-forming substrate 7.

The inhalation device 1 may include a closure 15 for retaining the aerosol-forming substrate 7 inside the receptacle 5.

The atomizer 11 includes one or more heating elements (not illustrated) to provide heat to the aerosol-forming substrate 7 to generate the vapor or aerosol.

The inhalation device 1 may include an aerosol chamber 17 for receiving the generated aerosol, an outlet 19 and a flow channel 21 extending between the aerosol chamber 17 and the outlet 19. The atomizer 11, for example, defines or includes the aerosol chamber 17. The outlet 19 is defined by a mouthpiece 22 of the inhalation device 1 that is in fluid communication with the aerosol chamber 17 via the flow channel 21.

The inhalation device 1 includes one or more air inlets 23 formed in an outer surface of the inhalation device 1. The one or more air inlets 23 may be defined in or by a side wall or in or by a base of the housing 3 as shown schematically in FIG. 1.

When the closure 15 is in a closed position an air flow passage is defined allowing air to enter the receptacle 5 and the aerosol chamber 17 via the one or more air inlets 23. In use, air is drawn through the mouthpiece 22 by a user causing air to flow in through the air inlet 23, and pass through the air flow passage, the aerosol-forming substrate 7, the aerosol chamber 17 and the flow channel 21 and to the user through the mouthpiece 22. The vapor or aerosol generated by the atomizer 11 is transported by this air movement through the inhalation device 1 to outlet 19 and the user.

The inhalation device 1 also includes an energy source or supply 25 for powering the elements of the inhalation device 1. For example, the energy source or supply 25 can consist of or include a battery such as a rechargeable lithium ion battery.

The inhalation device 1 may also include a user display or a sound generator 27. The user display may for example comprise or consist of a LED or LCD display. The sound generator may for example comprise or consist of a speaker.

The aerosol-forming substrate 7 is able or configured to release volatile compounds when the substrate 7 is heated to form the aerosol or vapor. The aerosol-forming substrate 7 is, for example, completely contained inside the inhalation device 1 as for example shown in FIGS. 1 to 5.

Alternatively, the aerosol-forming substrate 7 can be partially contained inside the inhalation device 1.

The solid aerosol-forming substrate 7 can include, for example, only solid components, or may alternatively include both solid and liquid components.

The aerosol-forming substrate 7 may comprise a tobacco ingredient containing agent. A tobacco ingredient containing agent can be any compound, mixture, particle matter, and/or solution that contains and/or carries a constituent of tobacco, either artificially included or naturally contained in tobacco, e.g. tobacco, tobacco particles, tobacco flavor and/or nicotine.

The aerosol-forming substrate 7 may comprise, in addition to tobacco ingredient containing agent or instead of the tobacco ingredient containing agent, an inhalable agent. The inhalable agent can be any compound, mixture, particle matter and/or solution that may be inhaled, for example as a gas and/or aerosol, and it e.g. includes and/or carries at least one of a stimulant, e.g. caffeine, guarana and combinations thereof, and/or a flavor, e.g. menthol, natural and/or artificial plant flavors, saccharides, animal flavors, and combinations thereof. The inhalable agents can be included to have the same approximate proportion with the aerosol forming agent as found in conventional e-liquids well known to persons skilled in the art.

The aerosol-forming substrate 7 may further comprise an aerosol forming agent. An aerosol forming agent can be any compound, mixture and/or solution that is capable of forming an aerosol, e.g. when heated and/or in mixture with a tobacco ingredient containing agent and/or an inhalable agent. Examples of suitable aerosol forming agents are glycerol, glycol derivatives, sebacate esters, and/or mixtures thereof, without being limited thereto.

The aerosol-forming substrate 7 may preferably comprise or consist of a mousse or a tobacco mousse containing the vaporizable substance 9.

The mousse tobacco mousse may comprise a foam. The foam is preferably used in a way that it is not heated to a temperature where it is combusted and only the vaporizable substance 9 contained therein is evaporated.

The foam can comprise the tobacco ingredient containing agent. In one exemplary embodiment, the foam comprises, for example, the tobacco ingredient containing agent and/or the inhalable agent, the aerosol forming agent, a foam stabilizing agent, and a foam forming agent.

The foam forming agent can include or consist of, for example, a non-protein containing polysaccharide.

The foam stabilizing agent is not particularly limited as long as it can stabilize the foam to some extent after formation. The foam stabilizing agent of the present foam can be selected from the group consisting of cellulose gum, hydroxyalkylated carbohydrates, derivatives thereof, e.g. salts thereof, preferably alkali metal salts thereof, e.g. sodium and/or potassium salts thereof, and mixtures thereof.

The foam may, for example, comprise or consist of an open pored foam to be understood as a foam, wherein the gas pockets in the foam connect with each other, contrary to a closed-cell foam, wherein the gas is in discrete pockets, each completely enclosed by a foam material.

The foam is preferably not smokable. The foam is preferably used in a way that it is not heated to such temperature where it is combusted, but only at least parts thereof are evaporated, for example particularly at least essentially the aerosol forming agent and further preferably at least a part of the tobacco ingredient containing agent and/or the inhalable agent, further preferably also essentially the tobacco ingredient containing agent and/or the inhalable agent.

In the foam, at least some parts of the tobacco ingredient containing agent and/or the inhalable agent preferably is, for example, adhered to the foam structure and/or absorbed by the foam structure formed essentially by the foam forming agent and the foam stabilizing agent, so that it can be easily released together with the aerosol forming agent upon heating. Also, it is possible that some parts of the tobacco ingredient containing agent and/or the inhalable agent are bound to the foam structure and the tobacco ingredient containing agent and/or the inhalable agent are ‘extracted’ during heating thereof, so that a flavor from the tobacco ingredient containing agent and/or the inhalable agent is released thereof together with the aerosol forming agent.

The tobacco ingredient containing agent and/or the inhalable agent can be configured in such a way that it is released upon heating together with the aerosol forming agent by only being essentially adsorbed to and/or absorbed in the foam structure.

The mousse can, for example, be one including a foam as defined in international patent application WO2018122375, the entire contents of which are fully incorporated herein by reference.

The inhalation device 1 also includes at least one controller 29 and a plurality of sensors, for example a first sensor 31, and at least a second sensor 33.

The first sensor 31 is configured to generate a first signal or to generate first data S1 associated with a presence or detection of at least one chemical substance in the generated aerosol. The second sensor 33 is configured to generate a second signal or generate second data S2 associated with a further characteristic of the generated aerosol, or of the aerosol-forming substrate 7, or of the vaporizable substance 9.

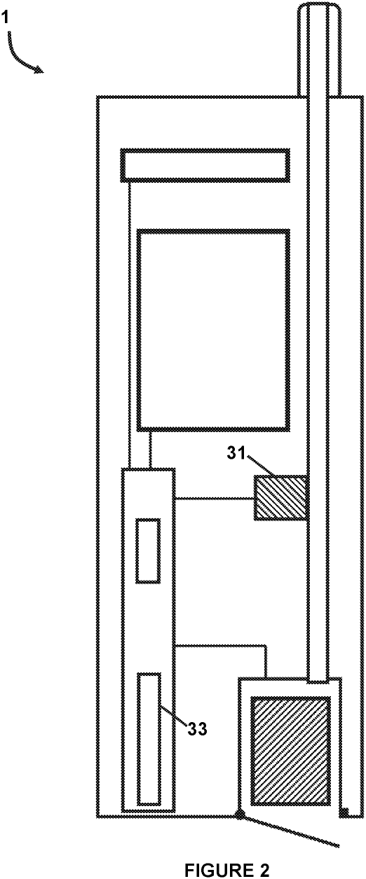

The second sensor 33 may alternatively be included partly or completely as part of the controller 29, as for example shown in FIG. 2.

The second sensor 33 is shown in a general manner in FIGS. 1 and 2. Specific elements or means for carrying out specific measurements by the second sensor 33 are not shown for ease of understanding in this general presentation of FIGS. 1 and 2.

The plurality of sensors, or the first sensor 31 and the second sensor 33 are configured to communicate with the controller 29 to respectively provide the first signal or first data S1 and the second signal or second data S2 thereto.

The controller 29 is in operative connection (electrically connected) with elements of the inhalation device 1. The controller 29 is configured to communicate signals or data to different elements of the inhalation device 1, and/or to receive signals or data from different elements of the inhalation device 1. Such elements include, for example, the plurality of sensors 31, 33, the energy source 25, the atomizer 11 and the user display or sound generator 27.

The controller 29 includes a calculator or processor configured to generate signals or data and to communicate these to different elements of the inhalation device 1. The calculator or processor is also configured to receive and process signals or data received from the different elements of the inhalation device 1. The calculator or processor can comprise or consist of, for example, a microcontroller, microprocessor, data processor, or electronic circuit.

The inhalation device 1 includes a memory 35 that may be a volatile or non-volatile memory (for example, semiconductor memory, HDD, or flash memory) configured to store or storing at least one or a plurality of programs or processor executable instructions. The programs or processor executable instructions comprise instructions permitting, for example, to control and command the inhalation device 1 operation and to control and command elements of the inhalation device 1.

The programs or processor executable instructions may comprise instructions permitting to generate signals or data to be communicated to different elements of the inhalation device 1 and permitting to receive and process signals or data received from different elements of the inhalation device 1.

The programs or processor executable instructions can include instructions permitting various different actions to be performed concerning analyzing data and determining an operation mode of the inhalation device 1 of the present disclosure.

The programs or processor executable instructions are provided to or obtained by the processer for execution.

The controller 29 is, for example, connected to the energy source 25 and to the atomizer 11 and configured to control the quantity of energy provided to the heating elements of the atomizer 11 and to control the quantity of vapor generated. The controller 29 is, for example, configured to reduce, increase or stop energy being provided to the heating elements and stop vapor generation.

The inhalation device 1 may, for example, include an activation button or air/fluid pressure sensor (not shown) that, when activated or pressed by a user, provides a signal to the controller 29 that is configured to subsequently provide energy to the atomizer 11 to generate a vapor. The air/fluid pressure sensor is, for example, activated by the user drawing air into the inhalation device 1 via the mouthpiece 22.

The controller 29 is, for example, connected to the user display or sound generator 27 and configured to determine visual or aural information presented to the user via the user display or sound generator 27.

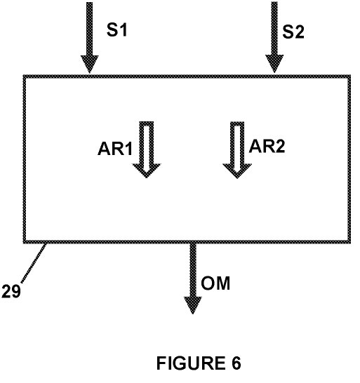

As shown schematically in FIG. 6, the controller 29 is configured to analyze both the first signal or first data S1 and the second signal or second data S2 separately in order to respectively determine a first analysis result AR1 and a second analysis result AR2. The controller 29 is also configured to determine, for example, an operation mode or action OM of the inhalation device 1 or to determine whether to alter an operation of the inhalation device 1 that is in progress based on both the first analysis result AR1 and the second analysis result AR2.

The first analysis result AR1 may, for example, concern user safety. The second analysis result AR2 may, for example, concern a determination or interrogation as to the potential source of the user safety indicator or alert determined by the first analysis result AR1, or the possible reason why the user safety indicator or alert has been triggered or determined.

The first analysis result AR1 may, for example, concern the determination of the presence of one or more chemical substances in the generated aerosol, or the presence of one or more chemical substances above one or more threshold levels, that may have a negative consequence for the user safety.

The second analysis result AR2 may, for example, concern aerosol-forming substrate identification, or vaporizable substance identification, or validation of the aerosol-forming substrate 7 or vaporizable substance 9 as suitable for the inhalation device.

The second analysis result AR2 may, for example, concern an operation condition or operation behavior of the inhalation device 1 and whether the operation condition or behavior is in conformity with a predetermined operation condition or behavior.

The second analysis result AR2 may, for example, concern a heating temperature of the aerosol-forming substrate 7, or the presence of a further substance in the generated vapor or in the environment surrounding the inhalation device 1.

The controller 29 is configured to determine an operation mode or action of the inhalation device 1 or to determine whether to alter an operation of the inhalation device 1 based on a conjunction of the first and second analysis results AR1, AR2. The state or value of the first analysis result AR1 and that of the second analysis result AR2 are each individually assessed or evaluated and considered in the determination of the operation mode by the controller 29. The controller is configured to determine an operation mode or action of the inhalation device 1 or to determine whether to alter an operation of the inhalation device 1 based on a separate evaluation of both the first and second analysis results AR1, AR2.

The controller 29 is configured to independently evaluate the first and second analysis results AR1, AR2.

The controller 29 is also configured to analyze the first signal or first data S1 independently or separately of the second signal or second data S2.

Alternatively, the controller 29 is configured to determine an operation mode or action OM of the inhalation device or to determine whether to alter an operation of the inhalation device 1 based on a combination of the first analysis result AR1 and the second analysis result AR2.

The controller 29 is, for example, configured to analyze the second signal or second data S2 to determine an identity of the aerosol-forming substrate 7 or of the vaporizable substance 9. The controller 29 is, for example, configured to analyze the second signal or second data S2 to validate the aerosol-forming substrate 7, vaporizable substance 9 or generated vapor as suitable for the inhalation device 1.

The controller 29 is, for example, configured to analyze the second signal or second data S2 to determine whether an operation condition or operation behavior of the inhalation device 1 is in conformity with a predetermined operation condition or operation behavior, or to determine whether an inhalation device element is located in a predefined position in or on the inhalation device 1.

The controller 29 is, for example, configured to determine an operation mode or action OM of the inhalation device 1 or to determine whether to alter the operation of the inhalation device 1 based on both (i) at least one chemical substance in the generated aerosol detected by the first sensor 31 and (ii) a determined identity of the aerosol-forming substrate 7 or of the vaporizable substance 9 determined using the second sensor 33, or validation or non-validation of the aerosol-forming substrate 7 or vaporizable substance 9 as being suitable for use by or in the inhalation device 1, and determined using the second sensor 33.

Alternatively or additionally, the controller 29 is, for example, configured to determine an operation mode or action OM of the inhalation device 1 or to determine whether to alter the operation of the inhalation device 1 based on both (i) at least one chemical substance in the generated aerosol detected by the first sensor 31 and (ii) an operation condition or operation behavior of the inhalation device 1 being in conformity with a predetermined operation condition or operation behavior, or an inhalation device element being located in a predefined position in or on the inhalation device 1.

The controller 29 is, for example, configured to determine (a) whether at least one chemical substance in the generated aerosol detected by the first sensor 31 is an authorized or conditionally/provisionally authorized chemical substance and (b) whether a determined identity of the aerosol-forming substrate 7 or the vaporizable substance 9 corresponds to an authorized aerosol-forming substrate 7 or authorized vaporizable substance 9, or whether the aerosol-forming substrate 7, vaporizable substance 9 or generated vapor is validated as being suitable for the inhalation device 1.

The controller 29 is, for example, configured to determine (a) whether at least one chemical substance in the generated aerosol detected by the first sensor 31 is an authorized or conditionally/provisionally authorized chemical substance and (b) whether an operation condition or operation behavior of the inhalation device 1 is in conformity with a predetermined operation condition or operation behavior, or an inhalation device element is located in a predefined position in or on the inhalation device 1.

The controller 29 is configured, for example, to permit operation of the atomizer 11 or to permit continued operation of the of the atomizer 11 when (i) the chemical substance in the generated aerosol detected by the first sensor 31 is determined to be an authorized or conditionally/provisionally authorized chemical substance and (ii) a determined identity of the aerosol-forming substrate 7 or of the vaporizable substance 9 or of the generated aerosol corresponds to an authorized aerosol-forming substrate, or authorized vaporizable substance, or the aerosol-forming substrate 7 or vaporizable substance is validated as being suitable for use in or by the inhalation device 1.

The controller 29 is configured, for example, to permit operation of the atomizer 11 or to permit continued operation of the of the atomizer 11 when (i) the chemical substance in the generated aerosol detected by the first sensor 31 is determined to be an authorized or conditionally/provisionally authorized chemical substance and (ii) an operation condition or operation behavior of the inhalation device 1 is in conformity with a predetermined operation condition or operation behavior, or an inhalation device element is located in a predefined position in or on the inhalation device 1.

The first sensor 31, can for example, be at least partially located in a flow path of the generated aerosol extending between the aerosol chamber 17 and the outlet 19. The first sensor 31, can for example, be located partially in the flow channel 21.

The first sensor 31 may comprise or consist solely of at least one combustion gas sensor configured to detect the presence, or a level or quantity, of at least one or a plurality of chemical substances or compounds in the generated aerosol or vapor. The level or quantity can be a value representing a concentration of a particular chemical substance or compound in the airflow, or can be an absolute value of the chemical substance or compound that is detected.

The chemical substance or compound can be, for example, one that may have potentially harmful consequences for the user or may negatively impact the user’s well-being, for example, when present at a measured level or quantity above a threshold level, or when existing in the presence or one or more additional chemical substances or compounds.

In a non-limiting example, the chemical substance may be carbon monoxide CO, or nitric oxide NO or nitrogen dioxide NO2 (NOx). The chemical substance may, for example, be one that is part of a carboxyl group or an aldehyde. The first sensor 31 can thus consist of or comprise a carbon monoxide detector, a nitric oxide detector or a nitrogen dioxide detector.

For example, in one embodiment, the chemical substance or compound may be one that alone is not a cause for concern but in the presence of one or more other substances (or in a quantity above a threshold level) may negatively impact the user’s well-being. Such a chemical substance requiring monitoring is referred herein to as a monitored or combination chemical substance or compound.

The controller 29 is configured to supervise the presence, or a level of one or more chemical substances or compounds using the signal or data S1 received from the first sensor 31 and processed by the controller 29. The controller 29 may receive data or the signal S1 containing an absolute value or a representative value from the first sensor 31. The controller 29 is configured to identify, and/or determine whether the value is an acceptable value or a value higher than one or more threshold values, for example, using a look-up table stored in memory 35.

In the case where the first sensor 31 is configured to determine the presence, or a level of a plurality of different chemical substances or compounds, the signal or data S1 includes an identifier, which the controller 29 is configured to process, to determine the identity of the associated chemical substance or compound, using for example associated information stored in the look-up table.

The controller 29 can be configured to immediately or quickly stop further operation of the inhalation device 1 relating to aerosol generation when the level or amount of the chemical substance or compound determined from the signal or data S1 is higher than an acceptable limit or threshold. Operation may, for example, be stopped or further prevented by the controller 29 shutting off energy supply to the heating elements of the atomizer 11. Conditional/provisional continued operation of the inhalation device 1 to generate the aerosol is not authorized. Further investigation is required for the controller 29 to determine an action to be taken in relation to the inhalation device 1 operation. This further investigation may for example, allow to determine the cause of the content of the signal or data S1 that shuts down vapor generation and provide it to the device user via, for example, the display 27 permitting corrective action by the user to be carried out.

The controller 29 can also be configured to immediately or quickly stop further operation of the inhalation device 1 relating to aerosol generation when a signal or data S1 associated with a presence of at least one (or a plurality of) forbidden or perilous chemical substance/compound in the generated aerosol is received by the controller 29. Such a signal or data S1, also requires no further investigation for the controller 29 to determine an action stopping vapor generation to be taken. Conditional/provisional continued operation of the inhalation device 1 to generate the aerosol is not authorized. Further investigation is required for the controller 29 to determine an action to be taken in relation to the inhalation device 1 operation. This further investigation may, for example, allow to determine the cause of the content of the signal or data S1 that shuts down vapor generation and provide it to the device user via, for example, the display 27 permitting corrective action by the user to be carried out.

The cause that shuts down vapor generation may, for example, be due to incorrect positioning of the substrate 7 in the inhalation device 1 or a non-identified or identifiable substrate 7 or substance 9 present in the device 1 and the user can be informed of such causes.

The first signal or first data S1 may thus comprise or consist of information requiring no further investigation by the controller 29 in relation to aerosol generation, that is, information that produces an immediate or direct decision by the controller 29 to stop aerosol generation or inhalation device 1 operation. Further investigation can be carried to determine the cause of the non-generation of vapor.

Dependent on the result of the second analysis results AR2 (or further analysis result or results AR) being favorable or satisfactory for operation or continued operation of the inhalation device 1, the controller 29 can be configured, for example, to allow operation or continued operation of the inhalation device 1 and/or aerosol generation or continued aerosol generation when the level or amount of the chemical substance or compound determined from the signal or data S1 is lower than an acceptable limit or threshold, and/or when a signal or data S1 associated with a non-presence of the at least one forbidden or perilous chemical substance/compound in the generated aerosol is received by the controller 29. The second analysis results AR2 (or further analysis result or results AR) can be, for example, the identification of the aerosol-forming substrate 7 or of the vaporizable substance 9, or the validation of the aerosol-forming substrate 7, substance 9 or the generated aerosol as suitable for the inhalation device 1, the determination of an operation condition or operation behavior of the inhalation device 1 being in conformity with a predetermined operation condition or operation behavior, or the determination of an inhalation device element being correctly located in a predefined position in or on the inhalation device 1. Exemplary embodiments of such second analysis results AR2 or further analysis result or results AR are further detailed below.

Of particular interest in the present disclosure is the first signal or first data S1 comprising or consisting of information requiring further investigation by the controller 29 when deciding to intervene or not in inhalation device 1 operation, for example, in relation to further or continued operation of the device 1 or in relation to further or continued aerosol generation. That is, information that forms or defines one factor in a multi-factor decision taken by the controller 29 on the basis of multiple factors (for example, at least two, or at least three) when deciding to intervene or not in inhalation device 1 operation, for example, in relation to further or continued operation of the device 1 or in relation to further or continued aerosol generation. The first signal or first data S1 defines a first factor signal or first factor data. Such a first signal or first data S1 may result in the controller placing the inhalation device operation under a conditional/ provisional operation or conditional/provisional continued operation.

In the case where, the first signal or first data S1 comprises or consists of information requiring further investigation (a first factor signal or first factor data) by the controller 29, the controller 29 determines the first analysis result AR1 based on the determination of, for example, the presence of a potentially undesired or under-surveillance chemical substance or compound, and/or the existence of a level or amount that is higher than a defined initial limit or threshold, or between a first and a second threshold value, the second threshold value being higher than the first and defining a limit beyond which the quantity of the chemical substance or compound is unacceptable. The chemical substance or compound is, for example, one that is acceptable or validated for use in the inhalation device 1, by the controller 29 or at least when present in a given quantity or in the absence or another particular chemical substance or compound.

The first analysis result AR1 can, for example, be defined as a positive analysis result requiring further investigation in the case where the controller 29 determines the presence of an undesired or under-surveillance chemical substance or compound, or when the level or amount is higher than the defined limit or threshold, or between the first and second threshold values. Otherwise, the first analysis result AR1 will be considered a negative analysis result and the controller 29 continues to process newly received signals or data S1 without taking any action or change in operation mode OM of the inhalation device 1, for example, in relation to aerosol generation.

The controller 29 is configured to additionally take into account the signal or data S2 received from at least one additional sensor, for example, the second sensor 33 to determine second analysis results AR2 and to determine an operation mode or action OM of the inhalation device 1 or to determine whether to alter the operation of the inhalation device 1.

In particular, the controller 29 is configured to do this when the first analysis result AR1 is a positive result requiring further investigation as mentioned previously, or when the inhalation device operation is under a conditional or provisional operation, or conditional or provisional continued operation.

The signal or data S2 defines a second factor signal or second factor data. The second factor signal or second factor data being independent of the first factor signal and first factor data, that is, these first and second factors are independently taken into account by the controller 29 to determine separate analysis results AR for the controller 29 to determine a decision relating to intervening in the operation of the inhalation device 1.

The above example is disclosed in relation to first and second sensors 31, 33, however, further signals or data S provided by additional sensors each defining addition factors may be also independently taken in account by the controller 29 with the mentioned first and/or second factors when the controller takes a decision.

The second sensor 33, for example, provides a signal or data S2 that may, for example, permit to determine the cause of the first analysis result AR1 coming from the first sensor or combustion gas sensor 31, or permit to modify device 1 operation to remove the cause of the first analysis result AR1 without any change or interruption of aerosol generation, or to permit to modify device 1 operation, or to protect the device user and/or the device 1. Additional sensors, in addition to the second sensor 33, can also provide a signal or data S that may permit the above possibilities.

The first 31 and/or second sensors 33 or the plurality of sensors, for example, operate and provide signals or data S when the inhalation device 1 or atomizer 11 is generating an aerosol, or alternatively when the inhalation device 1 or atomizer 11 has stopped generating an aerosol as the generated vapor or part thereof may still remain in the inhalation device 1.

The second sensor 33 is, for example, not a power sensor or energy supply sensor that determines whether power or energy is currently being supplied to one or more elements of the inhalation device 1.

The at least one additional sensor or second sensor 33 may, for example, comprise or consist solely of a capacitance sensor, or an optical emission sensor or a photo-sensor. The at least one additional sensor or second sensor 33 may for example, comprise or consist solely of a temperature sensor, or a chemical sensor, or a mass sensor, or a pH sensor, or a Hall-sensor, or a humidity sensor.

These listed sensors are provided as non-limiting exemplary sensors and other sensors can additionally or alternatively be included. The inhalation device 1 may include one or more additional sensors forming, for example, third, fourth sensors etc for providing a third signal or third data S3 (or third factor signal or third factor data) to determine a third analysis result AR3, and for providing a fourth signal or fourth data S4 (or fourth factor signal or fourth factor data) to determine a third analysis result AR4. Such sensors may, for example, comprises or consist of any one of the sensors listed above or below and are not limited to a total of four which is simply provided as an example.

Each sensor may provide an independent factor signal or factor data that are each independently taken in account by the controller 29.

The inhalation device 1 may thus include a plurality of sensors. The device 1 includes programs or processor executable instructions permitting the controller 29 to take into account each of the signals or data S to determine a plurality of analysis results AR, where each analysis result AR is taken into account by the controller 29 when determining an operation mode or action OM of the inhalation device 1 or when determining whether or how to alter the operation of the inhalation device 1. The pre-stored data contents of, for example, the look-up table is used or processed by the controller 29 to take a decision concerning device operation. The look-up table contains data associated with the information or data provided by the signals or data S that is processed by the controller 29 to determine a plurality of analysis results AR. The look-up table also includes data associated with the analysis results AR that is processed by the controller 29 to determine an operation mode or action OM of the inhalation device 1 or whether or how to alter the operation of the inhalation device 1. Combinations of different analysis results AR may determine a different operation mode or action OM of the inhalation device 1 that the controller 29 implements, or may determine that alteration of operation of the inhalation device 1 is not required based on current signal or data analysis. This allows the inhalation device to reliably determine an operation mode or action OM or to determine that alteration of operation of the inhalation device 1 is not required

Alternatively, the device 1 includes programs or processor executable instructions permitting the controller 29 to take into account a sub-group of the signals or data S to determine the plurality of analysis results AR to be considered by the controller 29 when taking a decision in relation to inhalation device 1 operation. The sub-group may be dynamically formed based on the information contained in the signal or data S. The sensors are, for example, attributed an initial priority and the controller 29 is configured to assess their signal or data S in order of this initial priority listing and to use pre-stored information in the look-up table to change the listing priority of the remaining sensors as the signal or data S from different sensors in the list is processed. This allows the inhalation device to quickly and reliably determine an operation mode or action OM or to determine that alteration of operation of the inhalation device 1 is not required.

In FIG. 1, the additional or second sensor is shown in a general manner as sensor 33. FIG. 2 shows that this sensor may at least be partially included in the controller 29. It should, however, be understood that the inhalation device 1 may have a plurality of such additional sensors each providing at least one signal or data S permitting to determine a further analysis result AR to be taken into account by the controller 29 when the first analysis result AR1 is a positive result requiring further investigation.

For example, in the case where the second sensor permits to identify or validate the substrate 7 or substance 9 present in the inhalation device 1, and the third sensor is a temperature sensor and the fourth sensor is a Hall-effect sensor, the fourth sensor that is the Hall-effect sensor may be attributed priority over the third sensor in the case where the substrate 7 is identified as one that is structurally of complex shape which is more likely to be incorrectly positioned in the device 1.

Exemplary second sensors are now presented in more detail. It should however be understood that any of these exemplary second sensors may be included as an additional sensor in the inhalation device 1 of the present disclosure.

The second sensor 33 can, for example, be configured to directly contact the aerosol-forming substrate 7 to perform a measurement to generate the second signal or second data S2.

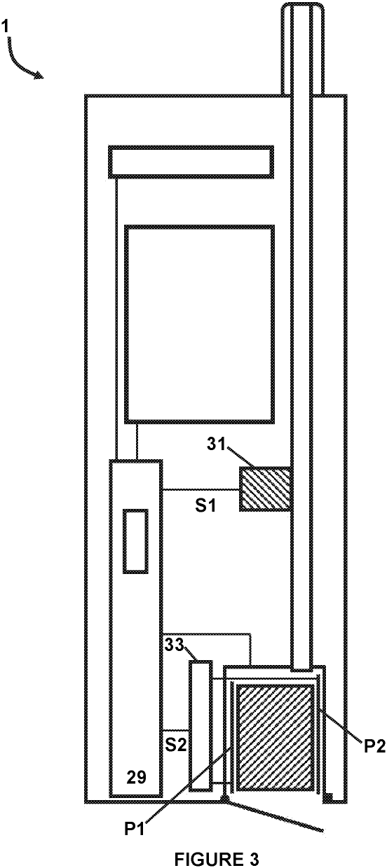

The second sensor 33 may, for example, comprise or consist of a capacitance sensor as schematically shown in the exemplary embodiment of FIG. 3. The capacitance sensor, for example, includes a first plate P1 and a second plate P2 each extending fully or partially along a side of the aerosol-forming substrate 7 and that each directly or indirectly contact potions of the aerosol-forming substrate 7 or substance 9 permitting a capacitance value of at least a portion of the aerosol-forming substrate 7 or the substance 9 to be measured by the second sensor 33. The measured value may be an absolute value or a value representing the capacitance value. This value is provided to the controller 29 as part of the second signal or second data S2.

The controller 29 is configured to assess an identity the aerosol-forming substrate 7 or the substance 9 contained thereon based on the value received as part of the second signal or second data S2 and using, for example, the look-up table stored in memory 35 that contains capacitance values attributed to a plurality of aerosol-forming substrates 7 and/or substances 9. The look-up table may also include information identifying one or more substances 9 contained in the identified substrate 7 allowing the substance 9 to be identified indirectly via identification of the aerosol-forming substrates 7 instead of by direct identification of the substance 9 via the measured capacitance value. Aerosol-forming substrates 7 or substances 9 can be, for example, structurally configured to provide predetermined capacitance values when measured thus permitting their identification or validation by the controller 29. Otherwise, the controller 29 determines that an unidentified or unidentifiable aerosol-forming substrate 7 or substance 9 is present.

A substrate 7 or substance 9 is, for example, determined to be one validated for use in the inhalation device 1 if the controller 29 determines that the substrate 7 or substance 9 is one that is stored or listed in the storage means 35 or look-up table.

Identification of a substrate 7 or substance 9 can, for example, allow the controller 29 to access further information stored in the storage means 35 or look-up table relating to the identified substrate 7 or substance 9 such as a preferred temperature profile or profiles for heating the substrate 7 or substance 9.

This signal or data S2 permits the controller 29 to determine the second analysis result AR2 which is additionally taken into account by the controller 29 in addition to the first analysis result AR1 to determine an operation mode or action OM of the inhalation device 1, or to determine whether to alter the operation of the inhalation device 1.

In the case where the second analysis result AR2 is the determination of an unidentified/unidentifiable or non-validated aerosol-forming substrate 7 or substance 9, the operation action OM can be, for example, an interruption of the vapor generation by the controller 29 stopping the supply of energy to the atomizer 11.

In the case where the second analysis result AR2 is the identification of an aerosol-forming substrate 7 or substance 9, or recognition of a substrate 7, substance 9 or generated vapor validated for use with the inhalation device 1, the operation action OM is determined, for example, by the controller 29 to be continued use of the inhalation device 1 with a limitation of vapor generation by limiting the supply of energy to the atomizer 11 to a predetermined level, for example, the current level or a level considered to be a precautionary safe level. The limitation may, for example, be removed depending on the outcome of a further determined analysis result AR. For example, the non-increase or maintenance of the measured level of the same substance or compound below the threshold level or between the first and second threshold levels over an immediate predetermined time duration (for example, 10 minutes or a duration of an average use of the inhalation device 1), or alternatively the absence or limited quantity of one or more other targeted or monitored chemical substances or compounds. This further analysis result AR may be determined, for example, using again the first sensor 31 or a signal or data S from a third sensor. Otherwise, the increase or non-maintenance, or the presence of such other chemical substances or compounds (or in a quantity above a threshold level) results for example in modification of the limitation state by the controller 29 to an interruption or stopping of vapor generation by the inhalation device 1, as previously described.

Alternatively, operation of the inhalation device 1 is allowed to continue unaltered and without any limitation until the detected level of the chemical substance or compound by the first sensor 31 extends beyond a further threshold level, such as the second threshold level.

The controller 29 is, for example, configured to permit operation or continued operation of the inhalation device 1 and more particularly the atomizer 11 when (i) the chemical substance in the generated aerosol detected by the first sensor 31 is determined to be an authorized chemical substance or provisionally/conditionally authorized chemical substance and (ii) an identity of the aerosol-forming substrate 7 or of the vaporizable substance 9 can be determined and thus corresponds to an authorized aerosol-forming substrate 7 or authorized vaporizable substance 9, or the aerosol-forming substrate 7, vaporizable substance 9 or generated vapor is one validated as suitable for use in or by the inhalation device 1 by the controller 29.

While the above described exemplary assessment and action is presented in relation to the capacitance sensor as the second sensor 33, it should be noted that this may equally to apply to other sensors described herein that permit the controller 29 to identify or validate as compatible substrates 7 or substances present in the inhalation device 1.

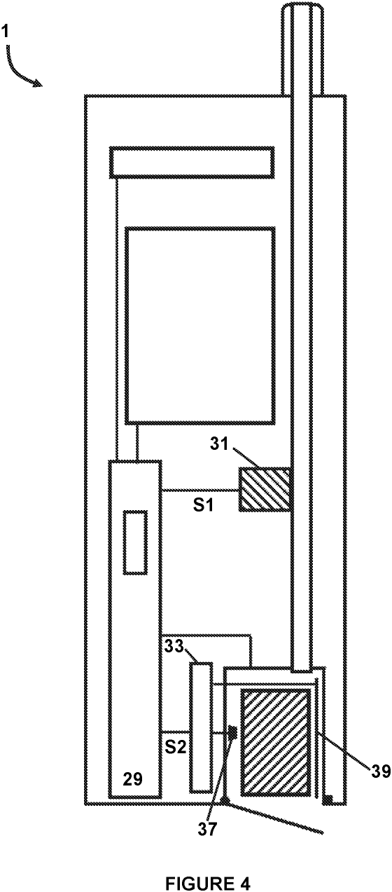

Additionally or alternatively, the second or additional sensor 33 may comprise or consist of an optical emission sensor or florescence sensor, as shown schematically in FIG. 4. The optical emission sensor includes an optical emitter 37 and an optical detector 39. The optical emitter 35 may comprise, for example, a laser diode or LED configured to emit electromagnetic radiation at one or more wavelengths that are absorbed by a material or optical marker configured to emit electromagnetic radiation when the material or marker absorbs the electromagnetic radiation provided by the optical emitter 35. The material or marker is included in the aerosol-forming substrate 7 or substance 9. The electromagnetic radiation emitted by the material or marker is, for example, at a shorter wavelength, and is detected by the optical detector 39 which is configured to provide the detected signal or data S2 to the controller 29. The optical detector 39 may include an optical filter to filter out the wavelength(s) emitted by the optical emitter 37 and may be configured to detect electromagnetic radiation at one or a plurality of wavelengths.

The material or optical marker can be configured to emit one or more specific optical signatures permitting the aerosol-forming substrate 7 or substance 9 to be identified by the controller 29. The aerosol-forming substance 9 may be identified directly by the material or optical marker emission, or indirectly through identification of the aerosol-forming substrate 7 via the material or optical marker emission, the controller 29 using the look-up table to determine the aerosol-forming substance 9 contained in the identified substrate 7.

The optical emitter 35 may, for example, be configured to emit in a UV wavelength or a wavelength in the visible or IR spectrum. The optical detector 39 may be configured to detect at one or more shorter wavelengths then the emission wavelength of the optical emitter 35.

In a non-limiting example, the optical emission sensor may, for example, comprise or consist solely of a fluorescence sensor. The aerosol-forming substrate 7 or the vaporizable substance 9 may thus contain fluorescing markers or chemicals to provide the optical emission. Other optical emission or luminesce processes may however be used.

The optical emission sensor 33 is thus configured to measure an optical emission signal emitted by at least a portion of the aerosol-forming substrate or the vaporizable substance under optical excitation and configured to provide the signal to the controller 29.

The optical emission sensor 33 may alternatively or additionally be configured to measure an optical emission signal emitted by the generated aerosol under optical excitation and configured to provide the signal to the controller 29. The optical emission sensor 33 in such a case is preferable located such that at least a part of the generated aerosol flows between the optical emitter 35 and the optical detector 39. Alternatively, the optical emission sensor 33 can be located along the flow channel 21 with the generated aerosol flowing between the optical emitter 35 and the optical detector 39. This permits for example to identify or determine the presence of the previously mentioned monitored chemical substance or compound, and to validate or not the generated aerosol for use (or continued use) in the inhalation device 1.

The optical emission sensor 33 provides the second signal or second data S2 to the controller 29 which is then processed by the controller 29, for example, in the manner explained previously.

The controller 29 is, for example, configured to analyze the optical emission signal provided to determine an identity of the aerosol-forming substrate 7 or the vaporizable substance 9, or to validate the generated aerosol. The controller 29 is configured, for example, to permit continued operation of the atomizer 11 when (i) the chemical substance in the generated aerosol detected by the first sensor 31 is determined to be an authorized chemical substance and (ii) the vaporizable substrate 7 or substance 9 is identified by the controller 29 using the signal or data S2 provided by the sensor 33 and thus an authorized substrate or substance, or the generated aerosol is validated for use, for example, one whose constituents does not contain a monitored chemical substance or compound and is thus an acceptable or authorized aerosol.

Additionally or alternatively, the second or additional sensor 33 may comprise or consist of a photo-sensor (or optical sensor) configured to measure an optical intensity signal transmitted through at least a portion of the aerosol-forming substrate 7 and/or the vaporizable substance 9, the photo-sensor being configured to provide the measured optical intensity signal to the controller 29 for processing.

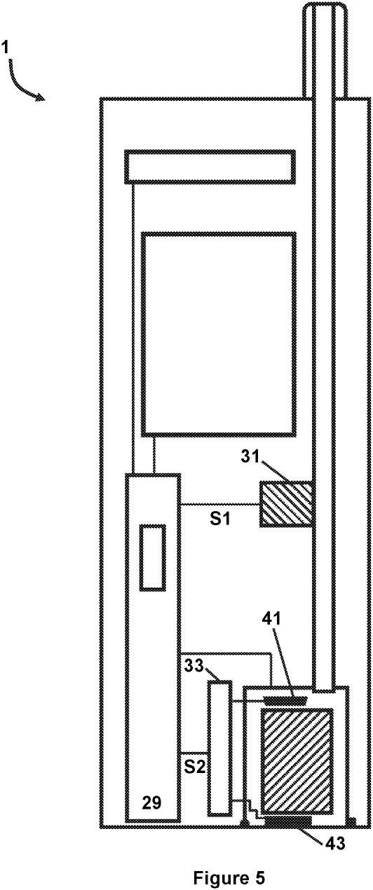

An exemplary photo-sensor is shown schematically in FIG. 5. The photo-sensor includes an optical emitter 41 and an optical intensity detector 43. The optical emitter 41 may comprise, for example, a laser diode or LED configured to emit electromagnetic radiation and an optical intensity detector 43 configured to provide a signal representing the optical intensity of the received electromagnetic radiation that has passed through at least a portion of the aerosol-forming substrate 7. The optical intensity detector 43 may, for example, comprise or consist of a light dependent resistor who resistance varies as a function of the light intensity received. The optical intensity detector 43 may, for example, comprise or consist of an array of image sensor (for example, a CMOS device) containing a plurality of pixels permitting one or more image patterns generated by the light passing through the substrate 7 to be detected. The optical intensity detector 43 can, for example, be attached to the closure 15 and the optical emitter 41 is located at an opposing end of the receptacle 5, the substrate 7 being positioned between the optical emitter 41 and the optical intensity detector 43.

The optical detector 43 may include an optical filter to filter out wavelength(s) different to that emitted by the optical emitter 41.

The optical detector 43 is configured to provide the detected signal or data S2 to the controller 29. The controller 29 may be configured to ignore values above or below a predetermined value which may correspond to a situation where no substrate 7 is present in the inhalation device 1 a direct exposure of on the optical detector 43 occurs.

The substrate 7 can be structurally configured to transmit a predetermined light intensity value or pattern permitting the controller 29 to identify the substrate 7, via, for example, the look-up table. The predetermined light intensity value or pattern received by the optical detector 43 can be, for example, formed by structuring the substrate 7 to include one or more openings and/or one or more material sections producing a desired transmission value and/or pattern. In one exemplary embodiment, the substrate 7 may, for example, from an optical transmission point of view be ringshaped where a center portion has a different transparency than the outer section and defines a particular transmission value (for example, within a predetermined range) or pattern for recognition by the controller 29.

The specific optical transmission values or patterns permit the aerosol-forming substrate 7 to be identified by the controller 29. The aerosol-forming substance 9 may be identified indirectly through identification of the aerosol-forming substrate 7 via the controller 29 using the look-up table to determine the aerosol-forming substance 9 contained in the identified substrate 7.

The photo-sensor sensor 33 provides the second signal or second data S2 to the controller 29 which is then processed by the controller 29, for example, in the manner explained previously.

The controller 29 is, for example, configured to permit operation of the atomizer 11 or permit continued operation of the of the atomizer when (i) the at least one chemical substance in the generated aerosol is determined to be an authorized chemical substance by the first sensor 31 or at least temporarily authorized and (ii) the the vaporizable substrate 7 and substance 9 is identified and thus is an authorized substrate 7 and substance 9.

As mentioned previously, the additional sensor or second sensor 33 may, for example, comprise or consist solely of a temperature sensor. The temperature sensor is configured to measure temperature values or values representing a temperature value in the atomizer 11, for instance a temperature value of the aerosol-forming substrate 7 or substance 9 or of the generated aerosol. The temperature sensor is configured to provide these values to the controller 29.

The controller 29 is configured, for example, to analyze the temperature values to determine whether the temperature values are in conformity with a predetermined temperature profile, for example, stored in the look-up table. The controller is configured, for example, to permit operation of the atomizer 11 or device 1 or permit continued operation of the atomizer 11 when (i) the at least one chemical substance in the generated aerosol is determined to be an authorized chemical substance by the first sensor 31 and (ii) the second analysis result AR2 based on the second signal or second data S2 determines that temperature values are in conformity with a predetermined temperature profile. Non-conformity with the predetermined temperature profile can, for example, be indicative of an operating problem in relation to vapor generation, for example, the presence of a non-compatible substrate 7 or substance 9 for use with the inhalation device 1 and result in the controller 29 interrupting the device or atomizer operation.

Alternatively, where the temperature sensor is an additional sensor, the controller 29 may be configured to determine an analysis result AR based on the signal or data S provided by the temperature sensor as a further analysis result (e.g. a third analysis result AR3) to determine that temperature values are in conformity with a predetermined temperature profile and to permit operation of the atomizer 11 or device or permit continued operation of the of the atomizer 11. In such a case, the controller 29 may for example take into account a first factor provided by the first sensor 31, a second factor provided by the second sensor that may be another sensor apart from the temperature sensor and a third factor provided by the temperature sensor. Non-conformity with the predetermined temperature profile can, for example, be indicative of an operating problem in relation to vapor generation even in the case where the substrate 7 or substance 9 is identified or validated for use with the inhalation device 1 which may however have become damaged or whose substance 9 content has been altered in a manner non-conform for use with the inhalation device 1, and result in the controller 29 interrupting the device or atomizer operation. Alternatively, the controller 29 may be configured to modify the energy supply to the atomizer 11 to attempt bring the measured temperature profile into conformity with the predetermined temperature profile and if non-conformity persists, the controller 29 interrupts the device or atomizer operation.

Similarly, the additional sensor or second sensor 33 may, for example, comprise or consist solely of a mass sensor. The mass sensor is configured to measure mass values or values representing mass values of the aerosol-forming substrate 7 and the vaporizable substance 9, and to provide the values to the controller 29. The controller 29 is configured to analyze the mass values to determine whether a predetermined change in mass has occurred during operation or heating of the aerosol-forming substrate 7, using for example data stored in the look-up table. The controller 29 is, for example, configured to permit operation of the atomizer 11 or permit continued operation of the of the atomizer 11 when (i) the at least one chemical substance in the generated aerosol is determined by the first sensor 31 to be an authorized chemical substance and (ii) the mass values are in conformity with a predetermined change-in-mass profile. Non-conformity with the predetermined profile can, for example, be indicative of an operating problem in relation to vapor generation, for example, the presence of a non-compatible substrate 7 or substance 9 for use in the inhalation device 1 and result in the controller 29 interrupting the device or atomizer operation.

The mass value may, for example, be measured indirectly vis an electrical resistance measurement or a capacitance measurement carried out on the substrate 7, the electrical resistance or capacitance changing as the quantity of the aerosol-generating substance 9 contained on the substrate 7 changes when heated.

Similar to the temperature sensor, the mass sensor may alternatively be an additional sensor and the controller 29 may be configured to determine an analysis result AR based on the signal or data S provided by the mass sensor as a further analysis result (e.g. a third analysis result AR3) to determine that values are in conformity with the predetermined profile and to permit operation or continued operation or not of the atomizer 11. Non-conformity with the predetermined profile may be indicative of a damaged substrate 7 or an altered aerosol-forming substance 9 content.

The additional sensor or second sensor 33 may, for example, comprise or consist solely of a humidity sensor. The humidity sensor can for example, be at least partially located in a flow path of the generated aerosol extending between the aerosol chamber 17 and the outlet 19. The humidity sensor, can for example, be located partially in the flow channel 21. The humidity sensor is configured to measure a value representing a water vapor content in the generated vapor and to provide the value to the controller 29.

The controller 29 is configured, for example, to analyze the value to determine whether the humidity value is within a predetermined humidity range and thus a generated vapor validated for use in the inhalation device 1, for example, stored in the look-up table. The controller is configured, for example, to permit operation of the atomizer 11 or device 1 or permit continued operation of the atomizer 11 when (i) the at least one chemical substance in the generated aerosol is determined to be an authorized chemical substance by the first sensor 31 and (ii) the second analysis result AR2 based on the second signal or second data S2 determines that humidity value is within the predetermined range of values. Non-conformity within the predetermined range (for example, at a high humidity) can be indicative of non-ideal operating conditions for the inhalation device 1 and for vapor generation and the controller 29 is configured to interrupt the device 1 or atomizer 11 operation when the humidity value is above or not within the predetermined range of values.

Alternatively, where the humidity sensor is an additional sensor, the controller 29 may be configured to determine an analysis result AR based on the signal or data S provided by the humidity sensor as a further analysis result (e.g. a third analysis result AR3) to determine that the humidity value is within the predetermined range and to permit operation of the atomizer 11 or device or permit continued operation of the of the atomizer 11. In such a case, the controller 29 may for example take into account a first factor provided by the first sensor 31, a second factor provided by the second sensor that may be another sensor apart from the humidity sensor and a third factor provided by the humidity sensor. Non-conformity within the predetermined humidity value range can, for example, result in the controller 29 interrupting the device 1 or atomizer 11 operation. The controller 29 can be configured to inform the inhalation device user via the user display 27.

The additional sensor or second sensor 33 may, for example, comprise or consist solely of at least one or a plurality of Hall-effect sensors. The Hall-effect sensor or sensors can be, for example, located in the atomizer 11 or outside the atomizer at a periphery thereof. The substrate 7 includes one or a plurality of corresponding magnets that are detected by the Hall-effect sensor or sensors when located in proximity to a Hall-effect sensor. The Hall-effect sensor or sensors are configured to provide a detection indicator or signal to the controller 29 when a magnet of the substrate is located in proximity to the Hall-effect sensor. In the case, where a plurality of Hall-effect sensors is used, the controller is configured to receive a plurality of distinct detection indicator or signals from the plurality of sensors. In the absence of the detection indicator or signal or in the absence of at least one of the plurality of indicator or signals from the plurality of sensors, the controller 29 is configured to determine that the substrate 7 is incorrectly positioned in the inhalation device 1 or atomizer 11. Otherwise, controller 29 is configured to determine that the substrate 7 is correctly positioned in the inhalation device 1 or atomizer 11 after the reception of the detection indicator or signal, or of all the plurality of indicator or signals.

The controller 29 is configured, for example, to permit operation of the atomizer 11 or device 1 or permit continued operation of the atomizer 11 when (i) the at least one chemical substance in the generated aerosol is determined to be an authorized chemical substance by the first sensor 31 and (ii) the second analysis result AR2 based on the second signal or second data S2 determines that the substrate 7 is correctly positioned in the inhalation device 1. Incorrect positioning can result non-ideal operating conditions for the inhalation device 1 and for vapor generation and the controller 29 is configured to interrupt the device 1 or atomizer 11 operation when incorrect positioning is determined by the controller 29. The controller 29 can be configured to inform the inhalation device user of the incorrect positioning via the user display 27.

Alternatively, where the Hall-effect sensor or sensors form an additional sensor or sensors, the controller 29 may be configured to determine an analysis result AR based on the signal or data S provided by the Hall-effect sensor or sensors as a further analysis result (e.g. a third analysis result AR3) and to permit operation of the atomizer 11 or device 1 or permit continued operation of the of the atomizer 11 when a correct positioning of the substrate 7 is determined by the controller 29. The controller 29 may for example take into account a first factor provided by the first sensor 31, a second factor provided by the second sensor that may be another sensor apart from the Hall-effect sensor and a third factor provided by the Hall-effect sensor or sensors. Incorrect positioning of the substrate 7 results in the controller 29 interrupting the device 1 or atomizer 11 operation. The controller 29 can be configured to inform the inhalation device user of the incorrect positioning via the user display 27.

Similarly, the additional sensor or second sensor 33 may, for example, comprise or consist solely of a chemical sensor. The chemical sensor is configured to contact the substrate 7, to detect the presence of at least one chemical substance or compound on the substrate 7 and to provide a value to the controller 29 indicative of the presence or not of the chemical substance or compound. The controller 29 is configured to determine that the substrate 7 is one identified or validated for use with the inhalation device 1 when the presence of the at least one chemical substance or compound is determined by the chemical sensor. The controller 29 is configured to determine an action to be taken in relation to the inhalation device operation in the manner explained previously in relation to the second sensor 33 identifying or validating a substrate for use with the inhalation device 1. The controller 29 is, for example, configured to permit operation of the atomizer 11 or permit continued operation of the of the atomizer 11 when (i) the at least one chemical substance in the generated aerosol is determined by the first sensor 31 to be an authorized chemical substance and (ii) the substrate 7 is one identified or validated for use with the inhalation device 1 by the chemical sensor.

Alternatively or additionally, one or more chemical sensors may be included and configured to detect the presence of a further substance, for example a monitored substance as mentioned previously, in the generated vapor to determine whether the generated vapor is validated for use in the inhalation device 1. The sensor can for example, be at least partially located in a flow path of the generated aerosol extending between the aerosol chamber 17 and the outlet 19, for example, located partially in the flow channel 21.

Alternatively or additionally, one or more chemical sensors may be included and configured to detect the presence of a substance in the environment surrounding the inhalation device 1. The sensor can, for example, be located at or in proximity to an external surface of the inhalation device 1. The controller is configured to use this information to determine whether a substance detected in the vapor has an internal source within the device or has an external source in the surrounding device environment, and to inform the user accordingly via the display 27. In the case where there the detected substance is from an external source, the user can be informed that the device 1 should be brought to a new location for continued use of the device.

Similarly, the additional sensor or second sensor 33 may, for example, comprise or consist solely of a pH sensor. The pH sensor is configured to contact the substrate 7, to measure a value representing a pH value on the substrate 7 and to provide the value to the controller 29. The controller 29 is configured to identify the substrate 7 based on a pH value range in which the received value falls, by for example, consulting such information stored in the look-up table. A received value outside of the predetermined ranges results in the controller 29 determining that an unidentified or non-validated substrate 7 is present in the inhalation device. The controller 29 is configured to determine an action to be taken in relation to the inhalation device operation in the manner explained previously in relation to the second sensor 33 identifying or validating a substrate for use with the inhalation device 1. The controller 29 is, for example, configured to permit operation of the atomizer 11 or permit continued operation of the of the atomizer 11 when (i) the at least one chemical substance in the generated aerosol is determined by the first sensor 31 to be an authorized chemical substance and (ii) the substrate 7 is one identified or validated for use with the inhalation device 1 by the pH sensor.

Similarly, the additional sensor or second sensor 33 may, for example, comprise or consist solely of a pH sensor. The pH sensor is configured to contact the substrate 7, to measure a value representing a pH value on the substrate 7 and to provide the value to the controller 29. The controller 29 is configured to identify the substrate 7 based on a pH value range in which the received value falls, by for example, consulting such information stored in the look-up table. A received value outside of the predetermined ranges results in the controller 29 determining that an unidentified or non-validated substrate 7 is present in the inhalation device. The controller 29 is configured to determine an action to be taken in relation to the inhalation device operation in the manner explained previously in relation to the second sensor 33 identifying or validating a substrate for use with the inhalation device 1. The controller 29 is, for example, configured to permit operation of the atomizer 11 or permit continued operation of the atomizer 11 when (i) the at least one chemical substance in the generated aerosol is determined by the first sensor 31 to be an authorized chemical substance and (ii) the substrate 7 is one identified or validated for use with the inhalation device 1 by the pH sensor.

As mentioned previously, the second sensor 33, for example, provides a signal or data S2 that can possibly determine the cause of the first analysis result AR1 coming from the first sensor or combustion gas sensor 31. This may lead to the controller 29 taking action to stop the inhalation device 1 operation, permitting continued operation under surveillance or not, or permitting continued operation under a provisional limited operating regime. Additional sensors third sensors, fourth sensors etc, in addition to the second sensor 33, can also provide a signal or data S that can further investigate or possibly determine the cause of the first analysis result AR1 coming from the first sensor or combustion gas sensor 31. This additional signal or data S can also be taken into account by the controller 29 to determine an operation mode or action of the inhalation device 1 or to determine whether to alter an operation of the inhalation device 1.

The inhalation device 1 may thus include a plurality of sensors each providing independent factors taken into consideration by the controller 29 when determining an operation mode or action of the inhalation device 1 or when determining whether to alter an operation of the inhalation device 1.

The present disclosure additionally concerns an inhalation device control method for controlling the above-mentioned inhalation device 1. The method includes, for example, separately analyzing, by the controller 29, both the first signal or first data S1 and the second signal or second data S2 to determine first analysis results AR1 and second analysis results AR2. The method further includes determining an operation mode or action of the inhalation device 1 or determining whether to alter an operation of the inhalation device 1 based on both the first and second analysis results.

The method further includes determining an operation mode or action of the inhalation device 1 or determining whether to alter an operation of the inhalation device 1 based on both the first and second analysis results.

The method may further include separately analyzing, by the controller 29, one or more further signals or data S to determine one or more further analysis results AR used in addition to determine an operation mode or action of the inhalation device 1 or to determine whether to alter an operation of the inhalation device 1.

Implementations described herein are not intended to limit the scope of the present disclosure but are just provided to illustrate possible realizations.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments and be given the broadest reasonable interpretation in accordance with the language of the appended claims. The features of any one of the above described embodiments may be included in any other embodiment described herein.

1. An inhalation device comprising:
a receptacle for receiving an aerosol-forming substrate containing at least one vaporizable substance;
an atomizer configured to generate an aerosol from the aerosol-forming substrate;
a first sensor configured to generate a first signal or first data associated with a presence of at least one chemical substance in the generated aerosol;
at least a second sensor configured to generate a second signal or second data associated with a further characteristic of the generated aerosol or of the aerosol-forming substrate or of the at least one vaporizable substance; and
at least one controller configured to separately analyze both the first signal or first data and the second signal or second data to determine first and second analysis results, and configured to determine an operation action or operation mode of the inhalation device or to determine whether to alter an operation of the inhalation device based on both the first and second analysis results.

2. The inhalation device according to claim 1, wherein the at least one controller is configured to determine an operation action or mode of the inhalation device or to determine whether to alter an operation of the inhalation device based on a conjunction of the first and second analysis results.

3. The inhalation device according to claim 1, wherein the at least one controller is configured to determine an operation mode or action of the inhalation device or to determine whether to alter an operation of the inhalation device based on a separate evaluation of both the first and second analysis results.

4. The inhalation device according to claim 3, wherein the at least one controller is configured to independently evaluate the first and second analysis results.

5. The inhalation device according to claim 1, wherein the at least one controller is configured to analyze the first signal or first data independently of the second signal or second data.

6. The inhalation device according to claim 1, wherein the at least one controller is configured to determine an operation mode or action of the inhalation device or to determine whether to alter an operation of the inhalation device based on a combination of the first and second analysis results.

7. The inhalation device according to claim 1, wherein the at least one controller is configured to analyze the second signal or second data to determine an identity of the aerosol-forming substrate or of the vaporizable substance , or to validate the aerosol-forming substrate, substance or generated aerosol as suitable for the inhalation device.

8. The inhalation device according to claim 1, wherein the at least one controller is configured to analyze the second signal or second data to determine whether an operation condition or operation behavior of the inhalation device is in conformity with a predetermined operation condition or operation behavior, or to determine whether an inhalation device element is located in a predefined position in or on the inhalation device.

9. The inhalation device according to claim 1, wherein the at least one controller is configured to determine an operation mode or action of the inhalation device or to determine whether to alter the operation of the inhalation device based on both (i) the at least one chemical substance in the generated aerosol detected by the first sensor; and (ii) a determined identity of the aerosol-forming substrate or of the vaporizable substance determined using the second sensor, or validation or non-validation of the aerosol-forming substrate, substance or generated aerosol as suitable for the inhalation device or an operation condition or operation behavior of the inhalation device being in conformity with a predetermined operation condition or operation behavior, or an inhalation device element being located in a predefined position in or on the inhalation device.

10. The inhalation device according to claim 1, wherein the at least one controller is configured to permit operation of the atomizer or permit continued operation of the atomizer when (i) the at least one chemical substance in the generated aerosol detected by the first sensor is determined to be a conditionally or provisionally authorized chemical substance; and (ii) a determined identity of the aerosol-forming substrate or of the vaporizable substance corresponds to an authorized aerosol-forming substrate or authorized vaporizable substance, or the aerosol-forming substrate,vaporizable substance or generated aerosol is validated as being suitable for the inhalation device, or an operation condition or operation behavior of the inhalation device is in conformity with a predetermined operation condition or operation behavior, or an inhalation device element is located in a predefined position in or on the inhalation device.

11. The inhalation device according to claim 1, wherein the at least one controller is configured to determine (a) whether the at least one chemical substance in the generated aerosol detected by the first sensor is a conditionally or provisionally authorized chemical substance; and (b) whether a determined identity of the aerosol-forming substrate or the vaporizable substance corresponds to an authorized aerosol-forming substrate or authorized vaporizable substance, or whether the aerosol-forming substrate, aerosol-forming substance or generated aerosol is validated as being suitable for the inhalation device, or whether an operation condition or operation behavior of the inhalation device is in conformity with a predetermined operation condition or operation behavior, or an inhalation device element is located in a predefined position in or on the inhalation device.

12. The inhalation device according to claim 1, wherein the second sensor is configured to directly contact the aerosol-forming substrate to perform a measurement to generate the second signal or second data.

13. The inhalation device according to claim 1, wherein the first analysis result concerns user safety, and the second analysis result concerns aerosol-forming substrate identification, or vaporizable substance identification, or validation of the aerosol-forming substrate, substance or generated vapor as suitable for the inhalation device.

14. The inhalation device according to claim 1, wherein the first sensor comprises or consists solely of a combustion gas sensor configured to detect the presence of at least one chemical substance in the generated aerosol.

15. The inhalation device according to claim 1, wherein the second sensor comprises or consists solely of a temperature sensor, a chemical sensor, a mass sensor, a pH sensor, a photo-sensor, a Hall sensor, a capacitance sensor, an optical emission sensor, or a humidity sensor.

16. An inhalation device control method,
providing the inhalation device comprising: a receptacle for receiving an aerosol-forming substrate containing a vaporizable substance, an atomizer configured to generate an aerosol from the aerosol-forming substrate, a first sensor configured to generate a first signal or first data associated with a presence of at least one chemical substance in the generated aerosol, at least a second sensor configured to generate a second signal or second data associated with a further characteristic of the generated aerosol or of the aerosol-forming substrate or of the vaporizable substance; and at least one controller;
separately analyzing, by the at least one controller, both the first signal or first data and the second signal or second data to determine first and second analysis results; and
determining an operation mode or action of the inhalation device or determining whether to alter an operation of the inhalation device based on both the first and second analysis results.
